(19) United States
(12) Patent Application Publication
(10) Pub. No.: US 2011/0236465 A1
(43) Pub. Date: Sep. 29, 2011

(54) MELT-EXTRUDED FILM

(76) Inventors: Mark J. Hall, Midland, MI (US); Michael D. Read, Midland, MI (US); Uma Shrestha, Bay City, MI (US)

(21) Appl. No.: 13/035,187

(22) Filed: Feb. 25, 2011

Related U.S. Application Data
(60) Provisional application No. 61/317,896, filed on Mar. 26, 2010.

(51) Int. Cl. A61K 9/70; B29C 47/12

(52) U.S. Cl. 424/443; 264/177.17

(57) ABSTRACT

A mono-layer or multi-layer film wherein at least one of the layers has a thickness of at least 0.125 mm is produced from a melt-extruded polymer composition comprising a) a water-soluble polymer, b) an active ingredient and c) an adjuvant selected from the group consisting of mono- and disaccharides, sugar alcohols, low molecular weight water soluble polymers, and salts of cross-linked carboxymethylcellulose, with the proviso that the adjuvant c) is different from the water-soluble polymer a).

### CROSS-REFERENCE TO RELATED APPLICATION(S)

This application claims benefit of priority from U.S. Provisional Patent Application No. 61/317,896, filed Mar. 26, 2010, which application is incorporated by reference herein in its entirety.

### FIELD OF THE INVENTION

This invention relates to melt-extruded films and a process for producing them.

### BACKGROUND OF THE INVENTION

Active ingredients, such as drugs or pharmaceuticals, may be prepared in a tablet form to allow for accurate and consistent dosing. However, this form of preparing and dispensing medications has many disadvantages including that a large proportion of adjuvants that must be added to obtain a size able to be handled, that a larger medication form requires additional storage space, and that dispensing includes counting the tablets which has a tendency for inaccuracy. In addition, many persons have difficulty swallowing tablets. While tablets may be broken into smaller pieces or even crushed as a means of overcoming swallowing difficulties, this is not a suitable solution for many tablet or pill forms. For example, crushing or destroying the tablet or pill form to facilitate ingestion, alone or in admixture with food, may also destroy the controlled release properties.

As an alternative to tablets and pills, films may be used to carry active ingredients. However, historically films and the process of making drug delivery systems there from have suffered from a number of unfavorable characteristics that have not allowed them to be used in practice. U.S. Patent Application Publication No. 2005/037055 discusses in detail in paragraphs [0005]-[0012] disadvantages of known films, such as agglomeration of film components which leads to an inhomogeneous distribution of the active ingredient or non-uniform films, particularly if the films are relatively thick. Non-uniform films are caused by conventional techniques for drying aqueous polymer solutions to produce a film, wherein the surface water is immediately evaporated forming a polymer film or skin. Evaporation of remaining water vapor under the surface of the film results in repeated destruction and reformation of the film surface, which is observed as a “ripple effect” which produces an uneven film. To solve these problems US 2005/037055 suggests the production of rapid-dissolving film products comprising a water-soluble polyethylene oxide alone or in combination with a hydrophilic cellulosic polymer which is free of added plasticizer. Polymer, water, and an active or other component is formed into a sheet or film by coating, spreading, casting or drawing the multi-component matrix and drying the film from the bottom of the film to the top of the film. Alternatively the film is formed by extrusion. According to the examples of US 2005/037055 rapid dissolving thin films having a content of an active ingredient of less than 5% by weight were produced by roll coating. While the taught drying method may be useful to obtain a uniform film, US 2005/037055 does not address the problem of how to dissolve a thick film rapidly to achieve a fast release of an active ingredient comprised in the film.

There is still a need to provide a fast disintegrating or dissolving thick film. Producing a fast disintegrating or dissolving thick film would allow incorporating a large and controlled amount of active ingredient into the film which could be released fast. Fast release of a large and controlled amount of active ingredient is a long-felt need.

### SUMMARY OF THE INVENTION

One aspect of the present invention is a mono-layer or multi-layer film wherein at least one of the layers has a thickness of at least 0.125 mm and is produced from a melt-extruded polymer composition comprising a) a water-soluble polymer, b) an active ingredient and c) an adjuvant selected from the group consisting of mono- and disaccharides, sugar alcohols, low molecular weight water soluble polymers, and salts of cross-linked carboxymethylcellulose, with the proviso that the adjuvant c) is different from the water-soluble polymer a).

Another aspect of the present invention is a process for producing a melt-extruded film which comprises the steps of

i) blending a) a water-soluble polymer, b) an active ingredient and c) an adjuvant selected from the group consisting of mono- and disaccharides, sugar alcohols, low molecular weight water soluble polymers, and salts of cross-linked carboxymethylcellulose, with the proviso that the adjuvant c) is different from the water-soluble polymer a), and, if desired, d) an optional additive and

ii) subjecting the blend to melt-extrusion to produce a film of a thickness of at least 0.125 mm.

### DETAILED DESCRIPTION OF THE INVENTION

At least one of the layers of the mono-layer or multi-layer film is melt-extruded and has a thickness of at least 0.125 mm, preferably at least 0.15 mm, more preferably at least 0.20 mm, and generally up to 0.50 mm, more preferably up to 0.35 mm, most preferably up to 0.30 mm. Preferably the film is in the form of a melt-extruded mono-layer film having the above-mentioned thickness. It has been found that a short disintegration or dissolution time of melt-extruded films having the above-mentioned thickness can be achieved if an adjuvant selected from the group consisting of mono- and disaccharides, sugar alcohols, low molecular weight water soluble polymers, and salts of cross-linked carboxymethylcellulose is included in a composition to be extruded in addition to a water-soluble polymer and an active ingredient. The adjuvant c) is different from the water-soluble polymer a).

At least one of the layers of the mono-layer or multi-layer film is produced from a melt-extruded polymer composition which preferably comprises from 10 to 94 percent, more preferably from 15 to 80 percent, and most preferably from 20 to 70 percent of a water-soluble polymer a), preferably from 1 to 80 percent, more preferably from 10 to 60 percent, and most preferably from 20 to 40 percent of an active ingredient b) and preferably from 5 to 50 percent, more preferably from 10 to 40 percent, and most preferably from 20 to 30 percent of an adjuvant c), based on the total weight of the polymer composition.

The melt-extruded polymer composition can comprise optional additives d) which are different from the components a), b) and c) of the composition. The amount of the optional additives d) is generally from 0 to 50 percent, typically from 0 to 45 percent, more typically from 10 to 40 percent, based on the total weight of the melt-extruded polymer composition. The combined amount of the water-soluble polymer a), the active ingredient b) and the adjuvant c) is preferably at least 70 percent, more preferably at least 80 percent, and most preferably at least 90 percent, based on the total weight of the polymer composition. The melt-extruded polymer composition can comprise one or more of the water-soluble polymers a), one or more of the active ingredients b), one or more of the adjuvants c), and one or more of the optional additives d), however their total amount is generally within the above-mentioned ranges.

The water soluble polymer a) preferably has a solubility in water of at least 1 grams, more preferably at least 3 grams, most preferably at least 5 grams in 100 grams of distilled water at 25° C. and 1 atmosphere. The water-soluble polymer a) is preferably selected from one or more polysaccharides, gelatins, poly(amino acids), such as poly(aspartic acid) or poly(glutamic acid); polylactic acid or a salt of such a polymerized acid or one or more synthetic polymers selected from the group consisting of polyalkylene oxides, such as ethylene oxide homo- and copolymers having a weight average molecular weight of at least 10,000, and homo- and copolymers comprising in polymerized form an unsaturated acid or a salt thereof, such as acrylic acid, methacrylic acid, or a salt thereof, an unsaturated amide, such as acrylamide; a vinyl ester, a vinylalcohol, an acetate, such as vinylacetate; an alkylene imine, such as ethylene imine; an oxyethylene alkylether, a vinylpyrrolidone, vinyloxazolidone, vinylmethyloxazolidone, ethylene sulfonic acid, a vinylamine, vinylpyridine, an ethylenically unsaturated sulfate or sulfonate or a combination of one or more of these polymers.

The water-soluble polymer a) generally has a weight average molecular weight of at least 50,000 g/mol, preferably at least 60,000 g/mol, more preferably at least 80,000 g/mol. The preferred upper limit for the weight average molecular weight largely depends on the type of polymer. Generally the weight average molecular weight of the water-soluble polymer is up to 10,000,000 g/mol, preferably up to 8,000,000 g/mol, more preferably up to 5,000,000 g/mol. The weight average molecular weight can be determined by light scattering according to the Standard Test Method ASTM D-4001-93 (2006).

One preferred type of water-soluble polymer a) is a polysaccharide. Examples of polysaccharides include gum arabic, xanthan gum, gum karaya, gum tragacanth, gum ghatti, carrageenan, dextran, alginates, agar, gellan gum, gallactomannans such as guar gum, pectins, starches, starch derivatives, guar derivatives and xanthan derivatives. Starch derivatives, guar derivatives and xanthan derivatives are described in more detail in European patent EP 0 504 870 B, page 3, lines 25-56 and page 4, lines 1-30. Useful starch derivatives are for example starch ethers, such as hydroxypropyl starch or carboxymethyl starch. Useful guar derivatives are for example carboxymethyl guar, hydroxypropyl guar, carboxymethyl hydroxypropyl guar or cationized guar. Preferred hydroxypropyl guars and the production thereof is described in U.S. Pat. No. 4,645,812, columns 4-6. Preferred polysaccharides are cellulose esters or cellulose ethers. Preferred cellulose ethers are carboxy-C₁-C₃-alkyl celluloses, such as carboxymethyl celluloses; carboxy-C₁-C₃-alkyl hydroxy-C₁-C₃-alkyl celluloses, such as carboxymethyl hydroxyethyl celluloses; C₁-C₃-alkyl celluloses, such as methylcelluloses; C₁-C₃-alkyl hydroxy-C₁₋₃-alkyl celluloses, such as hydroxyethyl methylcelluloses, hydroxypropyl methylcelluloses or ethyl hydroxyethyl celluloses; hydroxy-C₁₋₃-alkyl celluloses, such as hydroxyethyl celluloses or hydroxypropyl celluloses; mixed hydroxy-C₁-C₃-alkyl celluloses, such as hydroxyethyl hydroxypropyl celluloses, or alkoxy hydroxyethyl hydroxypropyl celluloses, the alkoxy group being straight-chain or branched and containing 2 to 8 carbon atoms. Most preferably, the composition comprises a water-soluble cellulose ether, such as a methylcellulose with a methyl degree of substitution DS_{methoxyl} of from 1.2 to 2.2, preferably from 1.5 to 2.0, or a hydroxypropyl methylcellulose with a DS_{methoxyl} of from 0.9 to 2.2, preferably from 1.1 to 2.0 and a MS_{hydroxypropoxyl} of from 0.02 to 2.0, preferably from 0.1 to 1.2. Generally the weight average molecular weight of the polysaccharide is from 50,000 g/mol to 5,000,000 g/mol, preferably from 60,000 g/mol to 500,000 g/mol, more preferably from 80,000 g/mol to 300,000 g/mol.

Another preferred type of water-soluble polymer a) is a polyethylene oxide. The term “polyethylene oxide” as used herein includes homo- and copolymers of ethylene oxide. The ethylene copolymer may be a random copolymer produced by the polymerization of ethylene oxide mixed with at least one other oxide, such as 1,2-cyclohexene epoxide, 1,2-butene epoxide, allyl glycidyl ether, glycidyl methacrylate, epichlorohydrin, 1,3-butadiene diepoxide, styrene oxide, 4-vinyl-1-cyclohexene 1,2-epoxide, 4-(2-trimethoxysilylethyl)-1,2-epoxycyclohexene and 4-vinyl-1-cyclohexene diepoxide, preferably an alkylene oxide, such as propylene oxide, 1,2-butene epoxide, or isobutylene oxide. Other useful ethylene oxide copolymers are block copolymers produced by the sequential addition of ethylene oxide and at least one other alkylene oxide, in which nearly total consumption of the first monomer takes place prior to the addition of subsequent monomer(s). Alternatively, the ethylene oxide copolymer may comprise in copolymerized form ethylene oxide and another copolymerizable monomer, such as methyl acrylate, ethyl acrylate, a caprolactone, ethylene carbonate, trimethylene carbonate, 1,3-dioxolane, carbon dioxide, carbonyl sulfide, tetrahydrofuran, methyl isocyanate, or methyl isocyanide. Preferred ethylene oxide copolymers are copolymers of ethylene oxide with epichlorohydrin or copolymers of ethylene oxide with cyclohexene oxide. Ethylene oxide copolymers generally comprise at least about 50 mole percent, preferably at least about 70 mole percent, more preferably at least about 85 mole percent ethylene oxide units. The most preferred ethylene oxide polymers are ethylene oxide homopolymers. The polyethylene oxide preferably has a weight average molecular weight of from 50,000 g/mol to 10,000,000 g/mol, more preferably from 60,000 g/mol to 8,000,000 g/mol, most preferably from 80,000 g/mol to 5,000,000 g/mol. Polyethylene oxides useful in the present composition are commercially available from The Dow Chemical Company. The average molecular weight of the polyethylene oxide employed will generally affect the processing conditions selected. A very high average molecular weight polyethylene oxide, such as greater than about 5,000,000 g/mol, will generally require higher processing temperature, torque and/or pressure in the extrusion process than a polyethylene oxide having an average molecular weight less than or equal to about 5,000,000 g/mol.

More preferably, the water-soluble polymer a) is an above-described cellulose ether or an above-described polyethylene oxide, a polyvinylpyrrolidone or a polymer comprising in polymerized form acrylic acid, methacrylic acid, a salt of acrylic acid or methacrylic acid, vinylacetate, ethylene imine, or an oxyethylene alkylether. Most preferably, an above-described cellulose ether or an above-described polyethylene oxide or a combination of a cellulose ether and a polyethylene oxide is utilized in the production of the film of the present invention.

A large variety of active ingredients can be included in the film of the present invention, preferably biologically active ingredients, particularly health-related biologically active ingredients, such as vitamins, herbals and mineral supplements, oral care ingredients and drugs, but also active ingredients not directly related to health, such as flavors, colors, taste masking compounds, cosmetically active ingredients, or ingredients active in agriculture. The active ingredient includes hydrophobic, hydrophilic and amphiphilic compounds. It is not necessary for the active ingredient to be soluble in any given component of the composition. The active ingredient may be dissolved, partially dissolved or suspended in the polymer matrix of the composition. The active ingredient should generally be stable during the melt extrusion process conditions used. By stable, it is meant that a significant portion of the active ingredient will not be significantly degraded or decomposed throughout the melt extrusion process. The resulting film is advantaged in that a given area of film can comprise a high concentration of active ingredient, thus fewer film strips are required to provide a therapeutic dose. Further, higher active ingredient concentration in the film provides faster availability of the active ingredient as less polymer must be dissolved before the film disintegrates.

The active ingredients which may be incorporated in the composition to be melt extruded may be used for treating indications such as, by way of example and without limitation, inflammation, gout, hypercholesterolemia, microbial infection, AIDS, tuberculosis, fungal infection, amoebic infection, parasitic infection, cancer, tumor, organ rejection, diabetes, heart failure, arthritis, asthma, pain, congestion, urinary tract infections, vaginal infection, seizure related disorder, depression, psychosis, convulsion, diabetes, blood coagulation, hypertension and birth control.

Examples of active ingredients that can be administered by the film of the present invention are, acebutolol, acetylcysteine, acetylsalicylic acid, acyclovir, alprazolam, alfacalcidol, allantoin, allopurinol, ambroxol, amikacin, amiloride, aminoacetic acid, amiodarone, amitriptyline, amlodipine, amoxicillin, ampicillin, ascorbic acid, aspartame, astemizole, atenolol, beclomethasone, benserazide, benzalkonium hydrochloride, benzocaine, benzoic acid, betamethasone, bezafibrate, biotin, biperiden, bisoprolol, bromazepam, bromhexine, bromocriptine, budesonide, bufexamac, buflomedil, buspirone, caffeine, camphor, captopril, carbamazepine, carbidopa, carboplatin, cefachlor, cefalexin, cefadroxil, cefazoline, cefixime, cefotaxime, ceftazidime, ceftriaxone, cefuroxime, selegiline, chloramphenicol, chlorhexidine, chlorpheniramine, chlortalidone, choline, cyclosporin, cilastatin, cimetidine, ciprofloxacin, cisapride, cisplatin, clarithromycin, clavulanic acid, clomipramine, clonazepam, clonidine, clotrimazole, codeine, cholestyramine, cromoglycic acid, cyanocobalamin, cyproterone, desogestrel, dexamethasone, dexpanthenol, dextromethorphan, dextropropoxiphene, diazepam, diclofenac, digoxin, dihydrocodeine, dihydroergotamine, dihydroergotoxin, diltiazem, diphenhydramine, dipyridamole, dipyrone, disopyramide, domperidone, dopamine, doxycycline, enalapril, ephedrine, epinephrine, ergocalciferol, ergotamine, erythromycin, estradiol, ethinylestradiol, etoposide, Eucalyptus globulus, famotidine, felodipine, fenofibrate, fenoterol, fentanyl, flavin mononucleotide, fluconazole, flunarizine, fluorouracil, fluoxetine, flurbiprofen, furosemide, gallopamil, gemfibrozil, gentamicin, Gingko biloba, glibenclamide, glipizide, clozapine, Glycyrrhiza glabra, griseofulvin, guaifenesin, haloperidol, heparin, hyaluronic acid, hydrochlorothiazide, hydrocodone, hydrocortisone, hydromorphone, ipratropium hydroxide, ibuprofen, imipenem, indomethacin, iohexol, iopamidol, isosorbide dinitrate, isosorbide mononitrate, isotretinoin, itraconazole, ketotifen, ketoconazole, ketoprofen, ketorolac, labetalol, lactulose, lecithin, levocarnitine, levodopa, levoglutamide, levonorgestrel, levothyroxine, lidocaine, lipase, imipramine, lisinopril, loperamide, lorazepam, lovastatin, medroxyprogesterone, menthol, methotrexate, methyldopa, methylprednisolone, metoclopramide, metoprolol, miconazole, midazolam, minocycline, minoxidil, misoprostol, morphine, multivitamin mixtures or combinations and mineral salts, N-methylephedrine, naftidrofuryl, naproxen, neomycin, nicardipine, nicergoline, nicotinamide, nicotine, nicotinic acid, nifedipine, nimodipine, nitrazepam, nitrendipine, nizatidine, norethisterone, norfloxacin, norgestrel, nortriptyline, nystatin, ofloxacin, omeprazole, ondansetron, pancreatin, panthenol, pantothenic acid, paracetamol, penicillin G, penicillin V, phenobarbital, pentoxifylline, phenoxymethylpenicillin, phenylephrine, phenylpropanolamine, phenytoin, piroxicam, polymyxin B, povidone-iodine, pravastatin, prazepam, prazosin, prednisolone, prednisone, bromocriptine, propafenone, propranolol, proxyphylline, pseudoephedrine, pyridoxine, quinidine, ramipril, ranitidine, reserpine, retinol, riboflavin, rifampicin, rutoside, saccharin, salbutamol, salcatonin, salicylic acid, simvastatin, somatropin, sotalol, spironolactone, sucralfate, sulbactam, sulfamethoxazole, sulfasalazine, sulpiride, tamoxifen, tegafur, teprenone, terazosin, terbutaline, terfenadine, tetracycline, theophylline, thiamine, ticlopidine, timolol, tranexamic acid, tretinoin, triamcinolone acetonide, triamterene, trimethoprim, troxerutin, uracil, valproic acid, vancomycin, verapamil, vitamin E, folinic acid and zidovudine.

Preferred active ingredients are ibuprofen (as racemate, enantiomer or enriched enantiomer), ketoprofen, flurbiprofen, acetylsalicylic acid, verapamil, paracetamol, nifedipine, captopril, omeprazole, ranitidine, tramadol, cyclosporin, trandolapril and therapeutic peptides.

Analgesics include opiates and opiate derivatives, such as oxycodone (available as Oxycontin®), ibuprofen, aspirin, acetaminophen, and combinations thereof that may optionally include caffeine.

Other preferred active ingredients for use in the present invention include anti-diarrheals such as immodium AD, anti-histamines, anti-tussives, decongestants, vitamins, and breath fresheners. Common drugs used alone or in combination for colds, pain, fever, cough, congestion, runny nose and allergies, such as acetaminophen, chlorpheniramine maleate, dextromethorphan, pseudoephedrine HCl and diphenhydramine may be included in the film compositions of the present invention.

Also contemplated for use herein are anxiolytics such as alprazolam (available as Xanax®); anti-psychotics such as clozopin (available as Clozaril®) and haloperidol (available as Haldol®); non-steroidal anti-inflammatories (NSAID's) such as dicyclofenacs (available as Voltaren®) and etodolac (available as Lodine®), anti-histamines such as loratadine (available as Claritin®), astemizole (available as Hismanal™), nabumetone (available as Relafen®), and Clemastine (available as Tavist®); anti-emetics such as granisetron hydrochloride (available as Kytril®) and nabilone (available as Cesamet™); bronchodilators such as Bentolin®, albuterol sulfate (available as Proventil®); anti-depressants such as fluoxetine hydrochloride (available as Prozac®), sertraline hydrochloride (available as Zoloft®), and paroxtine hydrochloride (available as Paxil®); anti-migraines such as Imigra®, ACE-inhibitors such as enalaprilat (available as Vasotec®), captopril (available as Capoten®) and lisinopril (available as Zestril®); anti-Alzheimer's agents, such as nicergoline; and CaH-antagonists such as nifedipine (available as Procardia® and Adalat®), and verapamil hydrochloride (available as Calan®).

Active antacid ingredients include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, tartaric acids and salts.

Cosmetic active ingredients may include breath freshening compounds like menthol, other flavors or fragrances, especially those used for oral hygiene, as well as actives used in dental and oral cleansing such as quaternary ammonium bases. The effect of flavors may be enhanced using flavor enhancers like tartaric acid, citric acid, vanillin, or the like.

Examples of the range of such nutritional supplements usable in the invention include, but are not limited to, Cherry extract, Ginkgo biloba extract, Kava Kava extract, Ginseng extract, Saw Palmetto extract, cranberry or blueberry extract, tomato extract, cordyceps sinensis extract, pomegranates, elderberries, as well as the entire berry family, strawberry, raspberry, cherry, black raspberry, boysenberry, etc., glucosamine sulfate, chromium picolinate, Milk thistle extract, Grape seed extract, Ma Huang extract, Co-enzyme Q10, water soluble vitamins such as vitamin C niacin, vitamin B1 and vitamin B12, and fat soluble vitamins such as vitamins A, D, E, and K, minerals such as calcium, magnesium and zinc, among others.

Examples of active ingredients which are particularly suitable for including in the polymer composition to be melt extruded are ibuprofen, ketoprofen, nifedipine, and acetaminophen.

The film of the present invention further comprises an adjuvant c) selected from the group consisting of mono- and disaccharides, sugar alcohols, low molecular weight water soluble polymers, and salts of cross-linked carboxymethylcellulose. The adjuvant c) is different from the water-soluble polymer a). Suitable mono- and disaccharides are galactose, fructose, dextrose, mannose, maltose, isomaltulose, lactose or sucrose. Lactose is preferred. Examples of sugar alcohols are mannitol, xylitol, sorbitol, adonitol, dulcitol, pentitols and hexitols. Mannitol is preferred. The sodium salt is the most preferred salt of cross-linked carboxymethyl cellulose. Suitable low molecular weight water soluble polymers are the types listed above for the water-soluble polymer a), however the water soluble polymers utilized as adjuvant c) have a weight average molecular weight of less than 40,000 g/mol, preferably less than 35,000 g/mol, more preferably less than 20,000 g/mol. The weight average molecular weight can be determined by light scattering according to the Standard Test Method ASTM D-4001-93 (2006).

The film of the present invention may comprise one or more optional additives d), such as one or more fillers, pigments, colorants, lubricants, plasticizers, stabilizers such as antioxidants, slip agents and anti-block agents. However, one advantage of the present invention is that it is not necessary to incorporate one or more lubricants or plasticizers or stabilizers or slip agents or anti-block agents in the polymer composition to be melt-extruded for preparing the film of the present invention.

The process for producing a melt-extruded film comprises the steps of i) blending the components a) one or more water-soluble polymers, b) one or more active ingredients, c) one or more of the above-described adjuvants and, if desired, d) one or more optional additives and ii) subjecting the blend to melt-extrusion to produce a film of a thickness of at least 0.125 mm.

The blends of a), b), c) and optionally d) described herein are generally melt-extrudable. As used herein, the term “melt-extrudable” refers to a compound or composition that may be melt-extruded, particularly hot-melt extruded. A hot-melt extrudable polymer composition is one that is sufficiently rigid at 25° C. and atmospheric pressure, when it is not in particulate form such as a powder or granules, but is capable of deformation or forming a semi-liquid state under elevated heat or pressure, that means at a temperature above 25° C. or a pressure above atmospheric pressure. Although the polymer composition utilized for producing the film of the present invention need not contain a plasticizer to render it hot-melt extrudable, a plasticizer may be included as an additional component. The plasticizer should be able to lower the glass transition temperature or softening point of the active composition in order to allow for lower processing temperature, extruder torque and pressure during the hot-melt extrusion process. Plasticizers also generally reduce the viscosity of a polymer melt thereby allowing for lower processing temperature and extruder torque during hot-melt extrusion. Useful plasticizers are, for example, cetanol, triglycerides, polyoxyethylene-polyoxypropylene glycol (Pluronic), triacetin or triethyl citrate. Plasticizers are advantageously included when a water-soluble polymer of very high molecular weight such as greater than about 5,000,000 g/mol, is employed.

The above-mentioned components a), b), c) and optionally d) are preferably mixed in the form of particles, more preferably in powdered form. The components a), b), c) and optionally d) may be pre-mixed before feeding the blend into a device utilized for melt-extrusion. Useful devices for melt-extrusion, specifically useful extruders, are known in the art. Alternatively, the components a), b), c) and optionally d) may be fed separately into the extruder and blended in the device before or during a heating step. Although in some embodiments of the invention the mixture or the components to be mixed in the extruder may contain liquid materials, dry feed is advantageously employed in the melt-extrusion process of the present invention. The composition or the components that has or have been fed into an extruder are passed through a heated area of the extruder at a temperature which will melt or soften the composition or at least one or more components thereof to form a blend throughout which the active ingredient is dispersed. The blend is subjected to melt-extrusion and caused to exit the extruder die using a take-up roll. Typical extrusion melt temperatures are from 50 to 210° C., preferably from 70 to 200° C., more preferably from 100 to 190° C. An operating temperature range should be selected that will minimize the degradation or decomposition of the active ingredient and other components of the composition during processing. The extruder used to practice the invention preferably is a commercially available model equipped to handle dry feed and having a solid conveying zone, one or multiple heating zones, and an extrusion die. It is particularly advantageous for the extruder to possess multiple separate temperature controllable heating zones. Single or multiple screw extruders, preferably twin screw extruders, can be used in the melt-extrusion process of the present invention. The gap of the extruder die is preferably from 0.40 to 1.5 mm, more preferably from 0.55 to 1.4 mm, most preferably from 0.64 to 1.2 mm. The die can have any shape known in the art, such as for example square, rectangular, or annular.

The molten or softened mixture preferably has a melt draw elongation of from 50 to 5000%, more preferably from 100 to 2500%, most preferably from 250 to 750%. The melt draw elongation is represented by the equation ((Vf−Vi)/Vi)*100, where Vi is the film velocity at the extruder die and Vf is the film velocity at the take-up roll. The take up roll, also designated as casting roll or chill roll, is the first surface that the molten formulation contacts after leaving the die. The roll rotation speed is controlled to provide the desired film thickness and drawdown rates from the extruded material.

The extrudate is molded, preferably drawn, to a film of the desired thickness, i.e., to a thickness of at least 0.125 mm. At least the preferred embodiments of the above-mentioned components a), b), c) and optionally d) in the above-mentioned weight ratios generally form a melt of sufficient melt strength that the extrudate can be drawn to a film at a draw-down ratio of from 1.2 to 10, preferably from 1.5 to 8, more preferably from 2 to 7. The term “draw-down ratio” as used herein is the ratio of the gap of the extruder die to the thickness of the drawn film at the take-up roll.

If a multi-layer film is to be produced, the molded film can be combined with other films layers while it is still warm or hot or after it has been cooled down. Alternately, a melt-extruded multi-layer film can be produced via coextrusion, wherein one or more of the layers are produced from the polymer composition comprising the above-mentioned components a), b), c) and optionally d).

The mono- or multi-layer film can be cut into dosage forms according to a manner known in the art.

The present invention is further illustrated by the following examples which are not to be construed to limit the scope of the invention. Unless otherwise mentioned, all parts and percentages are by weight.

### EXAMPLES

Film dissolution testing is performed according to the following procedure. The test is performed in a glass petri dish (70×50 mm). Film specimens are cut from the extruded strip into rectangles of 34 mm×22 mm. The actual thickness of each film specimen is measured prior to testing. 5 ml of deionized water (37° C.) is added to the dish. The film is then placed on top of this water. 20 ml additional deionized water of 37° C. is then added to the dish (25 ml, total). The timer is started when the last of the water is added. Film integrity is visually monitored. The dish is gently swirled every 10 seconds. ‘Disintegration Time’ is the time for the film to start breaking apart (any observable change in shape or size). ‘Dissolution Time’ is the time when the film completely dissolves (no fragments visible). Samples were measured in triplicate and averaged in order to determine an ‘Average Disintegration Time’ and ‘Average Dissolution Time’.

### Example 1

Component A is POLYOX WSR N-80 NF (Trademark of The Dow Chemical Company). This material is a polyethylene oxide polymer, with a molecular weight of 200,000 g/mol. Component B is ibuprofen (Spectrum Chemical). Component C is mannitol (SPI Polyols Inc.). These materials were blended at a 55/25/20 ratio (POLYOX WSR N-80 NF /ibuprofen/mannitol) for 10 minutes using a laboratory V-blender.

Film extrusion was performed using a Davis Standard extruder equipped with a general purpose screw of 1.25 inch diameter (32 mm) and a length/diameter ratio of 24/1. The extruder was outfitted with an 8 inch (203 mm) wide cast film die with a die gap of approximately 0.025 inch (0.64 mm). The extruded film was drawn away from the die and cooled using a vertical 3 roll stack. The steel casting rolls were controlled at 14.5° C. using a Mokon Compu-Mate 100 controller. The extruder setpoints were: barrel zone 1=70° C., barrel zone 2=140° C., barrel zone 3=150° C., die zone 1=150° C., die zone 2=150° C. The extruder screw rate was 25 rpm. The formulation was fed to the extruder at a rate of 2.5 kg/hour using a K-tron model KCLKT-20 feeder in gravimetric mode. A 0.262 mm thick film was produced. The take-up roll speed was 2 feet (0.6 m) per minute; the film width was 5.2 inch (132 mm).

Film dissolution was performed according to the test procedure described above. An average disintegration time of 64 seconds and an average dissolution time of 350 seconds were measured.

### Comparative Example

Component A is POLYOX WSR N-80 NF (Trademark of The Dow Chemical Company). This material is a polyethylene oxide polymer, with a molecular weight of 200,000 g/mol. Component B is ibuprofen (Spectrum Chemical). These materials were blended at a 75/25 ratio (POLYOX WSR N-80 NF/ibuprofen) for 10 minutes using a laboratory V-blender.

Film extrusion was performed using the same extruder as in Example 1. The extruder setpoints were: barrel zone 1=70° C., barrel zone 2=130° C., barrel zone 3=140° C., die zone 1=140° C., die zone 2=140° C. The extruder screw rate was 25 rpm. The formulation was fed to the extruder at a rate of 2.5 kg/hour using a K-tron model KCLKT-20 feeder in gravimetric mode. A 0.269 mm thick film was produced. The take-up roll speed was 2 feet (0.6 m) per minute; the film width was 4.8 inch (122 mm).

Film dissolution was performed according to the test procedure described above. An average disintegration time of 77 seconds and an average dissolution time of 546 seconds were measured.

The Table 1 summarizes the comparison of the film properties of the film of the present invention and the comparative film.

TABLE 1

| | | | | Average | |
|---|---|---|---|---|---|
| | Film | Melt Draw | Draw | Dis- | Average |
| | Thickness | Elongation | Down | integration | Dissolution |
| Sample | (mm) | (%) | Ratio | Time (sec) | Time (sec) |
| Example 1 | 0.262 | 305 | 3.00 | 64 | 350 |
| Comparative | 0.269 | 316 | 2.86 | 77 | 546 |

The results illustrate that a long sought after thick film with fast release of the active ingredient can surprisingly be achieved with a melt extruded polymer composition. Thick films comprising an active ingredient which exhibit faster dissolution can be produced by inclusion of the adjuvants described. Films with similar fabrication conditions that only contain the water soluble polymer a) and the active ingredient b) dissolve slower than is desired for effective delivery of the active ingredient.

What is claimed is:

1. A mono-layer or multi-layer film wherein at least one of the layers has a thickness of at least about 0.125 mm and is produced from a melt-extruded polymer composition comprising a) a water-soluble polymer, b) an active ingredient and c) an adjuvant selected from the group consisting of mono- and disaccharides, sugar alcohols, low molecular weight water soluble polymers, and salts of cross-linked carboxymethylcellulose, with the proviso that the adjuvant c) is different from the water-soluble polymer a).

2. The film of claim 1 wherein the water-soluble polymer a), the active ingredient b) and the adjuvant c) amount to at least about 80 percent, based on the total weight of the polymer composition.

3. The film of claim 1 wherein the amount of the adjuvant c) is from about 10 to about 40 percent, based on the total weight of the polymer composition.

4. The film of claim 1 wherein the adjuvant c) is a mono- or disaccharide or a sugar alcohol.

5. The film of claim 1 wherein the amount of the active ingredient b) is from about 10 to about 60 percent, based on the total weight of the polymer composition.

6. The film of claim 1 wherein the water-soluble polymer is a cellulose ether, a polyethylene oxide, a polyvinylpyrrolidone or a polymer comprising in polymerized form acrylic acid, methacrylic acid, a salt of acrylic acid or methacrylic acid, vinylacetate, ethylene imine, or an oxyethylene alkylether.

7. The film of claim 1 wherein the water-soluble polymer is a cellulose ether or a polyethylene oxide or a combination of a cellulose ether and a polyethylene oxide.

8. The film of claim 1 wherein the amount of the water-soluble polymer is from about 15 to about 80 percent, based on the total weight of the polymer composition.

9. The film claim 1 in the form of a mono-layer melt-extruded film.

10. A process for producing a melt-extruded film comprising the steps of
i) blending a) a water-soluble polymer, b) an active ingredient and c) an adjuvant selected from the group consisting of mono- and disaccharides, sugar alcohols, low molecular weight water soluble polymers, and salts of cross-linked carboxymethylcellulose, with the proviso that the adjuvant c) is different from the water-soluble polymer a), and, if desired, d) an optional additive and
ii) subjecting the blend to melt-extrusion to produce a film of a thickness of at least about 0.125 mm.

11. The process of claim 10 wherein the blend is subjected to melt-extrusion, caused to exit an extruder die and drawn to a film using a take-up roll at a draw-down ratio of from about 1.2 to about 10, wherein the draw-down ratio is the ratio of the gap of the extruder die to the thickness of the drawn film at the take-up roll.

12. The process of claim 11 wherein the blend is subjected to melt-extrusion and drawn to a film at a draw-down ratio of about 2 to about 7.

13. The process of claim 10 wherein the blend is subjected to melt-extrusion, caused to exit an extruder die and drawn to a film using a take-up roll to provide a film of a melt draw elongation of from about 50 to about 5000%, wherein the melt draw elongation=((Vf−Vi)/Vi)*100, where Vi is the film velocity at the extruder die and Vf is the film velocity at the take-up roll.

14. The process of claim 10 wherein the film of a thickness of at least about 0.125 mm is combined with one or more other films during or after melt-extrusion to produce a multi-layer film.
